(19) United States
(12) Patent Application Publication
(10) Pub. No.: US 2017/0000710 A1
(43) Pub. Date: Jan. 5, 2017

(54) Conditioning Shampoos Containing Anionic Surfactants, Glucamides, And Fatty Alcohols

(71) Applicant: CLARIANT INTERNATIONAL LTD, Muttenz (CH)

(72) Inventors: Peter KLUG, Grossostheim (DE); Carina MILDNER, Geisenheim (DE)

(73) Assignee: Clariant International Ltd., Muttenz (CH)

(21) Appl. No.: 15/100,039

(22) PCT Filed: Nov. 27, 2014
(86) PCT No.: PCT/EP2014/003175

§ 371 Date: May 27, 2016

(30) Foreign Application Priority Data
Nov. 29, 2013 (DE) 10 2013 018 000.7

(51) Int. Cl. A61K 8/46; A61K 8/37; A61K 8/34; A61Q 5/00; A61Q 5/02

(52) U.S. Cl. CPC A61K 8/46; A61Q 5/006; A61Q 5/02; A61K 8/463; A61K 8/342; A61K 8/37

(57) ABSTRACT

The invention relates to a composition, containing: at least one N-alkyl-N-acylglucamine as component A, at least one fatty alcohol as component B, at least one anionic surfactant as component C, optionally at least one betaine surfactant as component D, optionally at least one further surfactant as component E, water as component F, and optionally at least one further additive as component G, which composition can be used as a hair-washing agent.

The invention relates to a composition comprising N-alkyl-N-acylglucamines, fatty alcohols and anionic surfactants, to the use thereof in a hair care method, and to the use of the composition for improving the foaming behavior and/or for improving the conditioning effect. The invention relates further to a method for producing a composition according to the invention.

Care products for skin and hair care, comprising N-alkyl-N-acylglucamines, fatty alcohols and quaternary ammonium compounds having at least one long-chained alkyl or alkenyl group, for example behenyltrimethyl-ammonium chloride, stearyltrimethylammonium chloride, lauryltrimethyl-ammonium chloride, cetyltrimethylammonium chloride, distearyldimethyl-ammonium chloride, are known.

Also known are cosmetic compositions comprising fatty acid N-alkyl polyhydroxyalkylamides and fatty alcohols.

WO 97/47284 proposes cosmetic preparations for use in the hair and skin care field, which preparations comprise (a) esterquats and (b1) sorbitan esters, (b2) polyol poly-12-hydroxystearates and/or (b3) glycerides as well as optionally (c1) alkyl and/or alkenyl oligoglycosides and/or (c2) fatty acid N-alkyl polyhydroxyalkylamides. The compositions are distinguished by an improved softness in the hair and a particularly pleasant feel to the skin. Specifically, examples with fatty acid N-alkyl polyhydroxyalkylamides derived from coconut fatty acid are disclosed.

WO 94/21226 discloses detergent mixtures comprising polyhydroxy fatty acid amides, monomeric cationic surfactants, fatty alcohols and optionally oily substances, hair treatment agents comprising those mixtures, and the use of the mixtures for the production of hair treatment agents. Specifically, examples with C12/14 fatty acid N-alkyl polyhydroxyalkylamides are disclosed.

Finely divided, storage-stable emulsions comprising inter alia fatty acid N-alkyl polyhydroxyalkylamides and fatty alcohols are known, for example, from WO 97/06870. Preference is given thereby to fatty acid N-alkyl polyhydroxyalkylamides based on lauric acid or C12/14 coconut fatty acid.

WO 96/27366 discloses cosmetic and pharmaceutical compositions which comprise fatty acid N-alkylglucamides as O/W emulsifiers in combination with fatty alcohols as co-emulsifiers. There is no reference to a conditioning action in hair care.

Although good results are already achieved with known compositions, there is still room for improvement, in particular as regards use in shampoos.

Accordingly, the object was to develop compositions which are easy to formulate, have increased viscosity at 40° C. and exhibit improved temperature stability, increased creaminess of the foam as well as outstanding conditioning properties.

It has been found that the object is achieved by compositions comprising at least one N-alkyl-N-acylglucamine, at least one fatty alcohol and at least one anionic surfactant.

Accordingly, the invention provides a composition comprising:
at least one N-alkyl-N-acylglucamine as component A,
at least one fatty alcohol as component B,
at least one anionic surfactant as component C,
optionally at least one betaine surfactant as component D,
optionally at least one further surfactant as component E,
water as component F,
optionally at least one further additive as component G.

Compositions according to the invention are preferably silicone-free and/or free of cationic polymers and/or free of cationic surfactants.

Compositions according to the invention exhibit good thickening, in particular at 40° C.

In the case of hair care agents, compositions according to the invention have improved sensory properties, a very good hair conditioning effect and are biodegradable as well as based on renewable raw materials.

The N-alkyl-N-acylglucamines used according to the invention, in which glucamine preferably denotes a N-1-deoxysorbityl group, are particularly preferably N-alkyl-N-acylglucamines of formula (I)

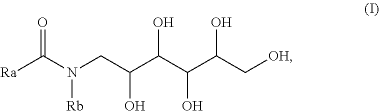

wherein in formula (I) RₐCO denotes a linear or branched, saturated or unsaturated C₆-C₂₂-acyl radical and R_{b} denotes a C₁-C₄-alkyl radical. Particularly preferably, R_{b} in formula (I) denotes a methyl radical (—CH₃) and RₐCO has the meaning given above.

Preferred N-alkyl-N-acylglucamines are compounds of formula (I) in which RₐCO denotes a C₁₆-C₁₈-acyl radical. Particular preference is given to N-alkyl-N-acylglucamines of formula (I) in which RₐCO denotes a C₁₆-C₁₈-acyl radical and R_{b} denotes a methyl radical.

The term C₁₆-C₁₈-acyl radical can generally be understood as meaning a compound having a C₁₆-acyl radical or C₁₈-acyl radical or a mixture thereof.

Preference is given as component A to saturated N-alkyl-N-acylglucamines of formula (I) wherein the acyl radical RₐCO is derived from palmitic acid, stearic acid, oleic acid or linoleic acid. Particular preference is given to N-alkyl-N-acylglucamines of formula (I) in which RₐCO is derived from palmitic acid, stearic acid, oleic acid or linoleic acid and R_{b} denotes a methyl radical.

As well as comprising the N-alkyl-N-acylglucamines of formula (I) in which RₐCO denotes a C₁₆-C₁₈-alkyl radical, the compositions can comprise small proportions of N-alkyl-N-acylglucamines derived from short-chained and/or long-chained fatty acids, in particular those which comprise C₁-C₄-acyl, C₆-, C₈-, C₁₀-, C₁₂-, C₁₄- and/or C₂₀-acyl.

Preferably, in such mixtures of component A, the content of N-alkyl-N-acylglucamines of formula (I) in which RₐCO denotes a C₁₆-C₁₈-acyl radical is 80% by weight, preferably 90% by weight, based on the content of component A.

In one embodiment there are used as N-alkyl-N-acylglucamines compounds of formula (I) in which RₐCO denotes a C12-C14-acyl radical. Particular preference is given to N-alkyl-N-acylglucamines of formula (I) in which RₐCO denotes a C₁₂-C₁₄-acyl radical and R_{b} denotes a methyl radical.

The term C₁₂-C₁₄-acyl radical can generally be understood as meaning a compound having a C₁₂-acyl radical or C₁₄-acyl radical or a mixture thereof.

In a particularly preferred embodiment, the N-alkyl-N-acylglucamine of a composition according to the invention is a mixture of at least one N-alkyl-N-acylglucamine of formula (I) wherein RₐCO denotes a C₁₆-acyl radical and at least one N-alkyl-N-acylglucamine of formula (I) wherein RₐCO denotes a C₁₈-acyl radical.

Particularly preferably, the ratio by weight of N-alkyl-N-acylglucamine of formula (I) wherein RₐCO denotes a C₁₆-acyl radical to N-alkyl-N-acylglucamine of formula (I) wherein RₐCO denotes a C₁₈-acyl radical is between 80:20 and 20:80, in particular between 65:35 and 35:65.

Preferably, the proportion of component A in a composition according to the invention is from 0.1 to 5.0% by weight, based on the composition, and particularly preferably from 0.2 to 1.0% by weight, based on the composition.

The N-alkyl-N-acylglucamines can be prepared, as described in EP 0 550 637 A1, by reacting the corresponding fatty acid esters or fatty acid ester mixtures with N-alkylglucamine in the presence of a solvent containing hydroxyl groups or alkoxyl groups. Suitable solvents are, for example, C1-C4-monoalcohols, ethylene glycol, propylene glycol, glycerol as well as alkoxylated alcohols. Preference is given to 1,2-propylene glycol. N-Alkylglucamine can be obtained, as likewise described in EP 0 550 637 A1, by reductive amination of glucose with alkylamine.

Suitable fatty acid esters which are reacted with the N-alkylglucamines to give N-alkyl-N-acylglucamines are generally the alkyl esters, in particular the methyl esters, which are obtained by transesterification from natural fats and oils, for example the triglycerides, or the triglycerides directly.

Suitable raw materials for the preparation of the fatty acid alkyl esters are, for example, coconut oil or palm oil.

Preferred N-alkyl-N-acylglucamines of formula (I) are accordingly also those compounds in which RₐCO is derived from coconut oil.

Coconut oil typically contains triglycerides which contain saturated fatty acid radicals derived from caprylic, lauric, capric, oleic, palmitic, stearic and myristic acid.

Coconut oil preferably comprises

a) from 40 to 55% by weight lauric acid,
b) from 10 to 20% by weight myristic acid,
c) from 8 to 12% by weight palmitic acid,
d) from 6 to 12% by weight oleic acid, and
h) from 0 to 36% by weight further fatty acids,
wherein the sum of the fatty acids bonded to the triglyceride is 100% by weight.

Particularly preferably, coconut oil comprises

a) from 40 to 55% by weight lauric acid,
b) from 10 to 20% by weight myristic acid,
c) from 8 to 12% by weight palmitic acid,
d) from 6 to 12% by weight oleic acid,
e) from 5 to 10% by weight decanoic acid,
f) from 4 to 10% by weight octanoic acid,
g) from 1 to 3% by weight stearic acid, and
h) from 0 to 26% by weight further fatty acids,
wherein the sum of the fatty acids bonded to the triglyceride is 100% by weight.

The compositions according to the invention comprise one or more fatty alcohols as component B, that is to say preferably unbranched or branched monoalcohols having an alkyl group having from 8 to 22 carbon atoms, particularly preferably from 16 to 22 carbon atoms, or a mixture thereof.

At such monoalcohols there come into consideration preferably cetyl alcohol, stearyl alcohol, behenyl alcohol and mixtures thereof, wherein cetyl alcohol, stearyl alcohol or a mixture thereof are particularly preferred.

Based on the total composition, the content of component B can be from 0.1 to 5.0% by weight. Preferably, the content of component B, based on the total composition, is from 0.2 to 1.0% by weight.

Preferably, the ratio by weight of N-alkyl-N-acylglucamine:fatty alcohol is from 20:80 to 80:20, wherein the ratio of N-alkyl-N-acylglucamine:fatty alcohol is preferably from 40:60 to 60:40.

The composition according to the invention further comprises at least one anionic surfactant as component C.

The compositions according to the invention comprise one or more anionic surfactants, preferably from the group of the alkyl sulfates and alkyl ether sulfates, particularly preferably in combination with betaine surfactants.

Preferred alkyl sulfates are the C₈-C₂₀-alkyl sulfates, in particular the linear C₈-C₂₀-alkyl sulfates, preferably in the form of their sodium, potassium or ammonium salts. Examples of alkyl sulfates are lauryl sulfate, coconut alkyl sulfate and tallow alkyl sulfate. Lauryl sulfate is particularly preferred.

Preferred alkyl ether sulfates are the C₈-C₂₀-alkyl ether sulfates, particular preference is given to the linear C₈-C₂₀-alkyl ether sulfates, in particular the alkyl glycol ether sulfates derived from the ethoxylated fatty alcohols, in the form of their sodium, potassium or ammonium salts. Examples of alkyl ether sulfates are lauryl ether sulfate, coconut alkyl ether sulfate, myristyl ether sulfate and tallow alkyl ether sulfate. Examples of glycol ether sulfates are lauryl triethylene glycol ether sulfate, coconut alkyl triethylene glycol ether sulfate and tallow alkylhexaethylene glycol ether sulfate. Particular preference is given to lauryl glycol ether sulfate, for example, lauryl diethylene glycol ether sulfate or lauryl triethylene glycol ether sulfate, especially in the form of the sodium salts. A preferred anionic surfactant is lauryl ether sulfate.

A particularly preferred anionic surfactant is sodium lauryl ether sulfate.

In a further preferred embodiment of the invention, the compositions comprise one or more N-acylamino acid surfactants as anionic surfactants. Within the scope of a preferred embodiment, the amino acid radical of such N-acyl-amino acid surfactants is selected from the group consisting of proteinogenic amino acids, their N-alkylated derivatives or mixtures thereof.

Particularly preferred as N-acyl-amino acid surfactants are acyl glycinates, acyl alaninates, acyl aspartates, acyl glutamates, acyl sarcosinates or mixtures thereof. Most particularly preferred are the N-acyl-amino acid surfactants selected from the group consisting of acyl glycinate, acyl aspartate, acyl glutamate, acyl sarcosinate and mixtures thereof.

Most particularly preferably, the N-acyl-amino acid surfactants consist of at least one C₈-C₂₂-acylated amino acid, in particular the N-alkylated derivatives thereof. The corresponding lauroyl or cocoyl derivatives of the amino acids are preferred.

Particular preference is therefore given to sodium cocoyl glycinate, potassium cocoyl glycinate, sodium lauroyl glycinate, potassium lauroyl glycinate, sodium cocoyl glutamate, sodium lauroyl glutamate, sodium cocoyl aspartate, sodium lauroyl aspartate and sodium lauroyl sarcosinate.

The proportion of component C in the composition according to the invention is preferably from 5.0% by weight to 20.0% by weight, based on the composition, particularly preferably from 8.0% by weight to 15.0% by weight, based on the composition.

Optionally and preferably, the compositions according to the invention comprise as component D, in addition to the at least one anionic surfactant, a betaine surfactant.

Betaine surfactants contain a cationic group, in particular an ammonium group, and an anionic group, which can be a carboxylate group, sulfate group or sulfonate group, in the same molecule. Suitable betaines are alkyl betaines such as coco-betaine or acylamidopropyl betaine. There can further be present as betaine surfactants also fatty acid alkylamidopropyl betaines, for example coconut acylamidopropyldimethyl betaine, C₁₂-C₁₈-dimethylamino hexanoates or C₁₀-C₁₈-acylamidopropanedimethyl betaines.

In a preferred embodiment of the invention, the compositions comprise one or more amidopropyl betaines of the general formula (II)

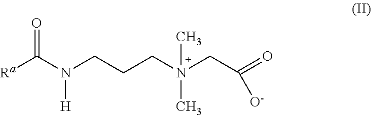

wherein R^{a} is a linear or branched saturated C₇-C₂₁-alkyl group or a linear or branched mono- or poly-unsaturated C₇-C₂₁-alkenyl group.

In a further embodiment of the invention, the compositions comprise one or more betaines of formula (III)

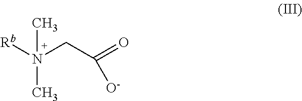

wherein R^{b} is a linear or branched₁ saturated C₈-C₂₂-alkyl group or a linear or branched mono- or poly-unsaturated C₈-C₂₂-alkenyl group.

In a further embodiment of the invention, the compositions comprise one or more sulfobetaines of formula (IV)

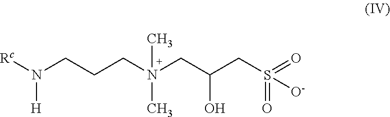

wherein R^{c} is a linear or branched saturated C₈-C₂₂-alkyl group or a linear or branched mono- or poly-unsaturated C₈-C₂₂-alkenyl group.

As well as comprising one or more alkyl sulfates and/or alkyl ether sulfates, the compositions preferably comprise one or more betaine surfactants selected from the group of compounds consisting of the amidopropyl betaines of formula (II), the betaines of formula (III) and the sulfobetaines of formula (IV).

In a particularly preferred embodiment of the invention, the compositions comprise one or more betaine surfactants selected from the amidopropyl betaines of formula (II).

In a further particularly preferred embodiment of the invention, the compositions comprise one or more betaine surfactants selected from the betaines of formula (III).

In a further particularly preferred embodiment of the invention, the compositions comprise one or more betaine surfactants selected from the sulfobetaines of formula (IV).

The radical R^{a} in the one or more amidopropyl betaines of formula (II) is preferably a linear or branched saturated C₇-C₁₇-alkyl group. Among the linear and branched saturated alkyl groups R^{a}, the linear saturated alkyl groups are preferred.

Particularly preferably, the amidopropyl betaines of formula (II) are cocamidopropyl betaines and the alkyl betaines of formula (III) are coco-betaine.

The radical R^{b} in the one or more betaines of formula (II) is preferably a linear or branched saturated C₈-C₁₈-alkyl group and particularly preferably a linear or branched saturated C12-C₁₈-alkyl group. Among the linear and branched saturated alkyl groups R^{b}, the linear saturated alkyl groups are preferred.

The radical R^{c} in the one or more sulfobetaines of formula (IV) is preferably a linear or branched saturated C₈-C₁₈-alkyl group and particularly preferably a linear or branched saturated C12-C₁₈-alkyl group. Among the linear and branched saturated alkyl groups R^{c}, the linear saturated alkyl groups are preferred.

As a further component E there can optionally be used cationic, non-ionic and/or amphoteric surfactants which are different from the surfactants mentioned above.

Suitable cationic surfactants are substituted or unsubstituted straight-chained or branched quaternary ammonium salts of the type R¹N(CH₃)₃X, R¹R²N(CH₃)₂X, R¹R²R³N(CH)₃X or R¹R²R³R⁴NX. The radicals R¹, R², R³ and R⁴ can preferably be, independently of one another, unsubstituted alkyl having a chain length of between 8 and 24 carbon atoms, in particular between 10 and 18 carbon atoms, hydroxyalkyl having from 1 to 4 carbon atoms, phenyl, C2- to C₁₈-alkenyl, C₇- to C₂₄-aralkyl, (C₂H₄O)ₓH, wherein x denotes from 1 to 3, one or more ester-group-containing alkyl radicals or cyclic quaternary ammonium salts. X is a suitable anion. Preference is given to (C₈-C₂₂)-alkyltrimethylammonium chloride or bromide, particularly preferably cetyltrimethylammonium chloride or bromide, di-(C₈-C₂₂)-alkyldimethylammonium chloride or bromide, (C₈-C₂₂)-alkyldimethylbenzylammonium chloride or bromide, (C₈-C₂₂)-alkyl-dimethylhydroxyethylammonium chloride, phosphate, sulfate, lactate, particularly preferably distearyldimethylammonium chloride, di(C₈-C₂₂)-alkylamidopropyltrimethylammonium chloride and methosulfate.

The amount of cationic surfactants in the compositions according to the invention can be up to 5% by weight, based on the total weight of the finished compositions.

The following compounds, for example, are suitable as non-ionic surfactants:

Polyethylene, polypropylene and polybutylene oxide condensates of alkylphenols. These compounds include the condensation products of alkylphenols having a C₆- to C₂₀-alkyl group, which can be either linear or branched, with alkene oxides. These surfactants are referred to as alkylphenol alkoxylates, for example alkylphenol ethoxylates.

Condensation products of aliphatic alcohols with from 1 to 25 mol of ethylene oxide. The alkyl or alkenyl chain of the aliphatic alcohols can be linear or branched, primary or secondary, and generally contains from 8 to 22 carbon atoms. Particular preference is given to the condensation products of C₁₀- to C₂₀-alcohols with from 2 to 18 mol of ethylene oxide per mol of alcohol. The alcohol ethoxylates can have a narrow (narrow range ethoxylates) or a broad homolog distribution of the ethylene oxide (broad range ethoxylates). Examples of commercially available non-ionic surfactants of this type are Tergitol® 15-S-9 (condensation product of a linear secondary C11-C15-alcohol with 9 mol of ethylene oxide), Tergitol® 24-L-NMW (condensation product of a linear primary C12-C24-alcohol with 6 mol of ethylene oxide with narrow molecular weight distribution). The Genapol® brands from Clariant also fall within this product class.

Condensation products of ethylene oxide with a hydrophobic base, formed by condensation of propylene oxide with propylene glycol. The hydrophobic part of these compounds preferably has a molecular weight between 1500 and 1800. The addition of ethylene oxide to the hydrophobic part leads to an improvement in the water solubility. The product is liquid up to a polyoxyethylene content of approximately 50% of the total weight of the condensation product, which corresponds to condensation with up to approximately 40 mol of ethylene oxide. Commercially available examples of this product class are the Pluronic® brands from BASF and the Genapol® PF brands from Clariant.

Condensation products of ethylene oxide with a reaction product of propylene oxide and ethylenediamine. The hydrophobic unit of these compounds consists of the reaction product of ethylenediamine with excess propylene oxide and generally has a molecular weight of from 2500 to 3000. Ethylene oxide is added to the hydrophobic unit up to a content of from 40 to 80% by weight polyoxyethylene and a molecular weight of from 5000 to 11,000. Commercially available examples of this class of compound are the Tetronic® brands from BASF and the Genapol® PN brands from Clariant.

Preferred non-ionic surfactants are fatty alcohol ethoxylates (alkyl polyethylene glycols); alkylphenol polyethylene glycols; fatty amine ethoxylates (alkylamino polyethylene glycols); fatty acid ethoxylates (acyl polyethylene glycols); polypropylene glycol ethoxylates (Pluronice); fatty acid alkanolamides (fatty acid amide polyethylene glycols); sucrose esters; sorbitol esters and sorbitan esters and their polyglycol ethers, as well as C₈-C₂₂-alkyl polyglucosides.

The amount of non-ionic surfactants in the compositions according to the invention (for example in the case of rinse-off products) is preferably in the range of from 0.1 to 10.0% by weight, particularly preferably from 0.5 to 5.0% by weight and more particularly preferably from 1.0 to 3.0% by weight.

The compositions according to the invention can further comprise amphoteric surfactants which are different from the betaines of component D. They can be described as derivatives of long-chained secondary or tertiary amines which have an alkyl group having from 8 to 18 carbon atoms and in which a further group is substituted by an anionic group which imparts water solubility, for example by a carboxyl, sulfate or sulfonate group. Preferred amphoteric surfactants are N—(C₁₂-C₁₈)-alkyl β-aminopropionates and N—(C₁₂-C₁₈)-alkyl β-iminodipropionates in the form of alkali and mono-, di- and tri-alkylammonium salts. Suitable further surfactants are also amine oxides. These are oxides of tertiary amines having a long-chained group of from 8 to 18 carbon atoms and two mostly short-chained alkyl groups having from 1 to 4 carbon atoms. Preference is given, for example, to the C₁₀- to C₁₈-alkyldimethylamine oxides and fatty acid amidoalkyl-dimethylamine oxides.

The amount of amphoteric surfactants is preferably from 0.5 to 20.0% by weight and particularly preferably from 1.0 to 10.0% by weight, based on the composition.

In a further embodiment, the compositions according to the invention additionally also comprise co-surfactants from the group of the fatty acid alkanolamides as foam-enhancing agents.

A composition according to the invention comprises water as component F.

The water content, based on the composition, is typically from 60 to 90% by weight, preferably from 70 to 88% by weight.

The compositions according to the invention can comprise as a further component G one or more additives, preferably from the group of the preservatives, fragrances, colorants, surfactants, cationic polymers, thickeners and gelling agents, pigments, antimicrobial and biogenic active ingredients, moisturizers, stabilizers, acids and/or alkalis.

Suitable preservatives are the preservatives listed in the relevant annex of the European cosmetics regulation, for example phenoxyethanol, benzyl alcohol, parabens, benzoic acid and sorbic acid; 1,3-bis(hydroxymethyl)-5,5-dimethylimidazoline-2,4-dione (Nipaguard® DMDMH), for example, is particularly suitable.

Fragrance or perfume oils can be used as fragrances. There can be used as fragrance or perfume oils individual fragrance compounds, for example synthetic products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether; the aldehydes include, for example, linear alkanals having from 8 to 18 carbon atoms, citral, citronellal, citronellyl oxyacetaldehyde, cyclamen aldehyde, hydroxy-citronellal, lilial and bourgeonal; the ketones include, for example, the ionones, alpha-isomethylionone and methyl cedryl ketone; the alcohols include anethol, citronellol, eugenol, geranion, linalool, phenylethyl alcohol and terpineol; and the hydrocarbons include mainly the terpenes and balsams. Preference is given to the use of mixtures of different fragrances, which together produce a pleasant fragrance note.

Perfume oils can also comprise natural fragrance mixtures as are obtainable from plant or animal sources, for example pine, citrus, jasmine, lily, rose or ylang-ylang oil. Essential oils of lower volatility, which are mostly used as flavor components, are also suitable as perfume oils, for example sage oil, chamomile oil, clove oil, melissa oil, mint oil, cinnamon leaf oil, lime blossom oil, juniper berry oil, vetiver oil, oliban oil, galbanum oil and ladanum oil.

The colorants and pigments, both organic and inorganic colorants, which are optionally present in the compositions according to the invention can preferably be selected from the corresponding positive list of the cosmetics regulation or the EC list of cosmetic colorants. There are advantageously used also pearlescent pigments, for example pearl essence (guanine/hypoxanthine mixed crystals from fish scales) and mother of pearl (ground mussel shells), monocrystalline pearlescent pigments such as, for example, bismuth oxychloride (BiOCl), layer-substrate pigments, for example mica/metal oxide, silver-white pearlescent pigments of TiO₂, interference pigments (TiO₂, different layer thickness), lustrous pigments (Fe₂O₃) and combination pigments (TiO₂/Fe₂O₃, TiO₂/Cr₂O₃, TiO₂/Prussian blue, TiO₂/carmine).

The amount of colorants and pigments in the compositions according to the invention is generally from 0.01 to 1.0% by weight, based on the total weight of the finished compositions.

Suitable cationic polymers are those known by the INCI name “polyquaternium”, in particular polyquaternium-31, polyquaternium-16, polyquaternium-24, polyquaternium-7, polyquaternium-22, polyquaternium-39, polyquaternium-28, polyquaternium-2, polyquaternium-10, polyquaternium-11, as well as polyquaternium 37&mineral oil&PPG trideceth (Salcare SC95), PVP-dimethylaminoethyl methacrylate copolymer, guarhydroxypropyltriammonium chloride, as well as calcium alginate and ammonium alginate. There can also be used cationic cellulose derivatives; cationic starches; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicone; copolymers of adipic acid and dimethylaminohydroxypropyldiethylenetriamine; polyaminopolyamide and cationic chitin derivatives, such as, for example, chitosan.

In one embodiment, a composition according to the invention comprises polyquaternium-7 as a cationic polymer.

In a further embodiment, a composition according to the invention comprises polyquaternium-10 as a cationic polymer.

In a further embodiment, a composition according to the invention comprises as a cationic polymer a galactomannan compound. Galactamannan 2-hydroxypropyltrimethylammonium chloride ether is preferred.

The compositions according to the invention can comprise one or more of the above-mentioned cationic polymers in amounts of from 0.1 to 5.0% by weight, preferably from 0.2 to 3.0% by weight and particularly preferably from 0.5 to 2.0% by weight, based on the finished compositions.

In a preferred embodiment, the compositions according to the invention are free of cationic polymers.

The desired viscosity of the compositions can be established by adding thickeners and gelling agents. There come into consideration preferably cellulose ethers and other cellulose derivatives (for example carboxymethylcellulose, hydroxyethylcellulose), starches and starch derivatives, sodium alginate, fatty acid polyethylene glycol esters, fatty acid alkanolamides, polyacrylamides or mixtures thereof. There can further be used crosslinked and uncrosslinked polyacrylates such as carbomers, sodium polyacrylates or sulfonic-acid-containing polymers such as ammonium acryloyldimethyltaurate/carboxyethyl acrylate crosspolymer.

The compositions according to the invention preferably comprise from 0.01 to 10.0% by weight, particularly preferably from 0.1 to 5.0% by weight, more particularly preferably from 0.2 to 3.0% by weight and most particularly preferably from 0.4 to 2.0% by weight, thickeners or gelling agents.

At antimicrobial active ingredients there are used cetyltrimethylammonium chloride, cetylpyridinium chloride, benzethonium chloride, diisobutylethoxy-ethyldimethylbenzylammonium chloride, sodium N-lauryl sarcosinate, sodium N-palmethyl sarcosinate, lauroylsarcosine, N-myristoylglycine, potassium N-laurylsarcosine, trimethylammonium chloride, sodium aluminum chlorohydroxy lactate, triethyl citrate, tricetylmethylammonium chloride, 2,4,4′-trichloro-2′-hydroxydiphenyl ether (triclosan), phenoxy-ethanol, 1,5-pentanediol, 1,6-hexanediol, 3,4,4′-trichlorocarbanilide (triclocarban), diaminoalkylamide, for example L-lysine hexadecyl amide, citrate heavy metal salts, salicylates, piroctose, in particular zinc salts, pyrithiones and heavy metal salts thereof, in particular zinc pyrithione, zinc phenol sulfate, farnesol, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, selenium disulfide and octopirox, iodopropynylbutylcarbamate, methylchloroiso-thiazolinone, methylisothiazolinone, methyldibromo glutaronitrile, AgCl, chloroxylenol, sodium salt of diethylhexylsulfosuccinate, sodium benzoate, as well as phenoxyethanol, benzyl alcohol, phenoxyisopropanol, parabens, preferably butyl-, ethyl-, methyl- and propyl-paraben, as well as the sodium salts thereof, pentanediol, 1,2-octanediol, 2-bromo-2-nitropropane-1,3-diol, ethylhexylglycerin, benzyl alcohol, sorbic acid, benzoic acid, lactic acid, imidazolidinylurea, diazolidinylurea, dimethyloldimethylhydantoin (DMDMH), sodium salt of hydroxymethyl glycinate, hydroxyethylglycine of sorbic acid, and combinations of these active ingredients.

The compositions according to the invention comprise the antimicrobial active ingredients preferably in amounts of from 0.001 to 5.0% by weight, particularly preferably from 0.01 to 3.0% by weight and more particularly preferably from 0.1 to 2.0% by weight, based on the finished compositions.

The compositions according to the invention can further comprise biogenic active ingredients selected from plant extracts, such as, for example, aloe vera, as well as local anesthetics, antibiotics, antiphlogistics, antiallergics, corticosteroids, sebostatics, Bisabolol®, Allantoin®, Phytantriol®, proteins, vitamins selected from niacin, biotin, vitamin B2, vitamin B3, vitamin B6, vitamin B3 derivatives (salts, acids, esters, amides, alcohols), vitamin C and vitamin C derivatives (salts, acids, esters, amides, alcohols), preferably in the form of the sodium salt of the monophosphoric acid ester of ascorbic acid or in the form of the magnesium salt of the phosphoric acid ester of ascorbic acid, tocopherol and tocopherol acetate, as well as vitamin E and/or derivatives thereof.

The compositions according to the invention can comprise biogenic active ingredients preferably in amounts of from 0.001 to 5.0% by weight, particularly preferably from 0.01 to 3.0% by weight and more particularly preferably from 0.1 to 2.0% by weight, based on the finished compositions.

There are available as the moisturizing substance, for example, isopropyl palmitate, glycerin glyceryl glucoside and/or sorbitol.

The compositions according to the invention are preferably adjusted to a pH in the range of from 2 to 12, preferably in the range of from 3 to 9, particularly preferably from 4 to 6.

The acids or alkalis used for adjusting the pH are preferably mineral acids, in particular HCl, inorganic bases, in particular NaOH or KOH, or organic acids, in particular citric acid or lactic acid.

The compositions according to the invention can further comprise oily substances. The oily substances can advantageously be selected from the groups of the triglycerides, natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids, or from the group of the alkyl benzoates, as well as natural or synthetic hydrocarbon oils.

There come into consideration triglycerides of linear or branched, saturated or unsaturated, optionally hydroxylated C₈-C₃₀-fatty acids, in particular vegetable oils, such as sunflower oil, corn oil, soybean oil, rice oil, jojoba oil, babusscu oil, pumpkin oil, grapeseed oil, sesame oil, walnut oil, apricot oil, orange oil, wheatgerm oil, peach kernel oil, macadamia oil, avocado oil, sweet almond oil, cuckoo flower oil, castor oil, olive oil, groundnut oil, rape oil and coconut oil, as well as synthetic triglyceride oils, for example the commercial product Myritol® 318. Hardened triglycerides are also preferred according to the invention. Oils of animal origin, for example beef tallow, perhydrosqualene, lanolin, can also be used.

A further class of preferred oily substances are the benzoic acid esters of linear or branched C₈₋₂₂-alkanols, for example the commercial products Finsolv® SB (isostearyl benzoate), Finsolv® TN (C₁₂-C₁₅-alkyl benzoate) and Finsolv® EB (ethylhexyl benzoate).

A further class of preferred oily substances are the dialkyl ethers having a total of from 12 to 36 carbon atoms, in particular having from 12 to 24 carbon atoms, such as, for example, di-n-octyl ether (Cetiol® OE), di-n-nonyl ether, di-n-decyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether, di-3-ethyldecyl ether, tert-butyl n-octyl ether, isopentyl n-octyl ether and 2-methylpentyl n-octyl ether as well as di-tert-butyl ether and diisopentyl ether.

A further class of preferred oily substances are hydroxycarboxylic acid alkyl esters. Preferred hydroxycarboxylic acid alkyl esters are full esters of glycolic acid, lactic acid, malic acid, tartaric acid or citric acid. Further esters of the hydroxycarboxylic acids which are suitable in principle are esters of β-hydroxypropionic acid, of tartronic acid, of D-gluconic acid, sugar acid, mucic acid or glucuronic acid. There are suitable as the alcohol component of these esters primary, linear or branched aliphatic alcohols having from 8 to 22 carbon atoms. The esters of C₁₂-C₁₅-fatty alcohols are particularly preferred. Esters of this type are available commercially, for example under the trade name Cosmacol® from EniChem, Augusta Industriale.

A further class of preferred oily substances are dicarboxylic acid esters of linear or branched C₂-C₁₀-alkanols, such as di-n-butyl adipate (Cetiol® B), di-(2-ethylhexyl) adipate and di-(2-ethylhexyl) succinate, as well as diol esters such as ethylene glycol dioleate, ethylene glycol diisotridecanoate, propylene glycol di-(2-ethyl hexanoate), propylene glycol diisostearate, propylene glycol dipelargonate, butanediol diisostearate and neopentyl glycol dicaprylate as well as diisotridecyl azelaate.

Symmetrical, non-symmetrical or cyclic esters of carbonic acid with fatty alcohols, glycerol carbonate or dicaprylyl carbonate (Cetiol® CC) are likewise preferred oily substances.

A further class of preferred oily substances are the esters of dimers of unsaturated C₁₂-C₂₂-fatty acids (dimer fatty acids) with monovalent linear, branched or cyclic C₂-C₁₈-alkanols or with polyvalent linear or branched C₂-C₆-alkanols.

A further class of preferred oily substances are hydrocarbon oils, for example those with linear or branched, saturated or unsaturated C₇-C₄₀-carbon chains, for example Vaseline, dodecane, isododecane, cholesterol, lanolin, synthetic hydrocarbons such as polyolefins, in particular polyisobutene, hydrogenated polyisobutene, polydecane, as well as hexadecane, isohexadecane, paraffin oils, isoparaffin oils, for example the commercial products of the Permethyl® series, squalane, squalene, and alicyclic hydrocarbons, for example the commercial product 1,3-di-(2-ethylhexyl)-cyclohexane (Cetiol® S), ozokerite and ceresin.

A composition according to the invention can further comprise silicone oils or silicone waxes. Silicone oils and silicone waxes that are available are preferably dimethylpolysiloxane and cyclomethicone, polydialkylsiloxanes R₃SiO(R₂SiO)ₓSiR₃, wherein R represents methyl or ethyl, particularly preferably methyl, and x represents a number from 2 to 500, for example the dimethicones obtainable under the trade names VICASIL (General Electric Company), DOW CORNING 200, DOW CORNING 225, DOW CORNING 200 (Dow Corning Corporation), as well as the dimethicones obtainable under SilCare® Silicone 41M65, SilCare® Silicone 41M70, SilCare® Silicone 41M80 (Clariant), stearyldimethylpolysiloxane, C₂₀-C₂₄-alkyl-dimethylpolysiloxane, C₂₄-C₂₈-lkyl-dimethylpolysiloxane, but also the methicones obtainable under SilCare® Silicone 41M40, SilCare® Silicone 41M50 (Clariant), also trimethylsiloxy silicates [CH₂)₃SiO)_{1/2}]ₓ[SiO₂]_{y}, wherein x represents a number from 1 to 500 and y represents a number from 1 to 500, dimethiconols R₃SiO[R₂SiO]ₓSiR₂OH and HOR₂SiO[R₂SiO]ₓSiR₂OH, wherein R represents methyl or ethyl and x represents a number up to 500, polyalkylarylsiloxanes, for example the polymethylphenylsiloxanes obtainable under the trade names SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty-acid-, alcohol-, polyether-, epoxy-, fluorine- and/or alkyl-modified silicone compounds, as well as polyether-siloxane copolymers.

However, a composition according to the invention is particularly preferably free of silicones.

The compositions according to the invention can further comprise film formers, which are selected, according to the intended application, from salts of phenylbenzimidazolesulfonic acid, water-soluble polyurethanes, for example C₁₀-polycarbamylpolyglyceryl ester, polyvinyl alcohol, polyvinyl-pyrrolidone copolymers such as PVP/hexanedecene or PVP/eicosene copolymer, for example vinylpyrrolidone/vinyl acetate copolymer, water-soluble acrylic acid polymers/copolymers or esters or salts thereof, for example partial ester copolymers of acrylic/methacrylic acid and polyethylene glycol ethers of fatty alcohols, such as acrylate/steareth-20 methacrylate copolymer, water-soluble cellulose, for example hydroxy-methylcellulose, hydroxyethylcellulose, hydroxypropylcellulose, water-soluble quaternium, polyquaternium, carboxyvinyl polymers, such as carbomers and salts thereof, polysaccharides, for example polydextrose and glucan, vinyl acetate/crotonate, for example obtainable under the trade name Aristoflex® A 60 (Clariant), as well as polymeric amine oxides, for example representatives obtainable under the trade names Diaformer Z-711, 712, 731, 751.

The compositions according to the invention can comprise one or more film formers in amounts of from 0.1 to 10.0% by weight, preferably from 0.2 to 5.0% by weight and particularly preferably from 0.5 to 3.0% by weight, based on the finished compositions.

Furthermore, a composition according to the invention can also comprise superfatting agents. There can be used as superfatting agents preferably lanolin and lecithin, non-ethoxylated and polyethoxylated or acylated lanolin and lecithin derivatives, polyol fatty acid esters, mono-, di- and tri-glycerides and/or fatty acid alkanolamides, wherein the latter at the same time serve as foam stabilizers, which are preferably used in amounts of from 0.01 to 10.0% by weight, particularly preferably from 0.1 to 5.0% by weight and more particularly preferably from 0.5 to 3.0% by weight.

Compositions according to the invention can also comprise pearlescent components. There are suitable as the pearlescent component preferably fatty acid monoalkanolamides, fatty acid dialkanolamides, monoesters or diesters of alkylene glycols, in particular ethylene glycol and/or propylene glycol or oligomers thereof, with higher fatty acids, such as, for example, palmitic acid, stearic acid and behenic acid, monoesters or polyesters of glycerol with carboxylic acids, fatty acids and metal salts thereof, ketosulfones or mixtures of the mentioned compounds. Particular preference is given to ethylene glycol distearates and/or polyethylene glycol distearates having on average 3 glycol units.

Where the compositions according to the invention comprise pearlescent compounds, the pearlescent compounds are present preferably in an amount of from 0.1 to 15.0% by weight and particularly preferably in an amount of from 1.0 to 10.0% by weight.

The total proportion of further additives as component G in the compositions according to the invention is preferably from 1.0 to 25.0% by weight and particularly preferably from 2.0 to 20.0% by weight, based on the composition.

The composition according to the invention particularly preferably does not comprise cationic polymers and/or cationic surfactants.

In a particularly preferred embodiment, the composition does not comprise silicones.

The compositions according to the invention preferably comprise:
from 0.1 to 5.0% by weight, based on the composition, of component A,
from 0.1 to 5.0% by weight, based on the composition, of component B,
from 5.0 to 20.0% by weight, based on the composition, of component C,
from 0 to 10.0% by weight, based on the composition, of component D.

The compositions according to the invention particularly preferably comprise:
from 0.2 to 1.0% by weight, based on the composition, of component A,
from 0.2 to 1.0% by weight, based on the composition, of component B,
from 8.0 to 15.0% by weight, based on the composition, of component C,
from 1.0 to 5.0% by weight, based on the composition, of component D.

In a preferred embodiment, a composition according to the invention comprises:

at least one N-alkyl-N-acylglucamine of formula (I) wherein RₐCO denotes a C₁₆-acyl radical,
at least one N-alkyl-N-acylglucamine of formula (I) wherein RₐCO denotes a C₁₈-acyl radical,
at least one monoalcohol as the fatty alcohol, in particular cetyl alcohol,
at least one lauryl ether sulfate, in particular sodium lauryl ether sulfate, as the anionic surfactant,
at least one betaine surfactant, in particular cocamidopropyl betaine,
no silicone, no cationic surfactant and no cationic polymer.

The compositions according to the invention are preferably shampoos for greasy hair, dry hair, damaged hair, hair with dandruff, a color shampoo, baby shampoo or sports shampoo.

The invention further provides a method of treating and caring for the hair with a composition according to the invention. In the method according to the invention, the hair is brought into contact with a composition according to the invention. The composition can be used as a rinse-off or leave-on product.

The invention further provides the use of the composition according to the invention for improving the foaming behavior, for example of a shampoo, and/or for improving the hair conditioning effect.

The invention further provides a method for producing a composition according to the invention. In the method according to the invention, components A, B, C, F and optionally components D, E and/or G are brought into contact with one another.

The examples which follow serve to explain the invention but without limiting it thereto.

### EXAMPLES

The N-alkyl-N-acylglucamines described in the following were prepared according to EP 0 550 637 from the corresponding fatty acid methyl esters and N-acyl glucamide in the presence of 1,2-propylene glycol as solvent and were obtained in the form of a solid consisting of active substance, that is to say N-alkyl-N-acylglucamine, and 1,2-propylene glycol (all data in % by weight).

TABLE 1
Preparation examples for N-alkyl-N-acylglucamine

| | | Active | 1,2- | |
|---|---|---|---|---|
| Preparation | | substance | Propylene | Melting point |
| Example | Methyl ester | (%) | glycol | (° C.) |
| 1 | C_{12/14} | 90 | 10 | 85 |
| | (C₁₂: 70%; | | | |
| | C₁₄: 30%) | | | |
| 2 | C_{16/18} | 80 | 20 | 65 |
| | (C₁₆: 60%; | | | |
| | C₁₈: 40%) | | | |

The melting point was determined by means of a Kofler hot bench.

The following test formulations were prepared, the percentages indicate the content of active substance in % by mass, based on the total composition. In the following table, in each case only the surfactants are mentioned.

Test formulations:

| | |
|---|---|
| Sodium lauryl ether sulfate (SLES), 2 EO | 8.0 or 9.0% |
| (Genapol LRO liq., Clariant) | |
| Cocamidopropyl betaine (CAPB), (Genagen CAB 818, | 3.0 or 3.5% |
| Clariant) | |
| C 12/14 glucamine according to Preparation Example 1 | 0; 0.5 or 1.0% |
| (90% in 1,2-propylene glycol) or | |
| C16/18 glucamine according to Preparation Example 2 | |
| (80% in 1,2-propylene glycol) | |
| Cetyl alcohol (Lanette 16, BASF) | 0 or 0.5% |
| Polyquaternium-7 (SalCare Super 7, BASF) | 0.2% |
| Sodium benzoate | 0.1% |
| Sodium salicylate | 0.1% |
| Perfume | 0.5% |
| pH = 5.0 | |

The compositions were evaluated as shampoos by a test panel (5 persons, home use test). The profile of the composition in question was evaluated in comparison with the standard (Example 1): −=negative, 0=satisfactory, +=good, ++=very good, and was additionally described descriptively (Table 2).

The viscosities were measured by means of a model DV II Brookfield viscometer, using the spindles from the RV spindle set at 20 revolutions/minute and 20° C. Spindles 1 to 7 from the RV spindle set were used. Under these measuring conditions, spindle 1 is chosen for viscosities not exceeding 500 mPa·s, spindle 2 for viscosities not exceeding 1000 mPa·s, spindle 3 for viscosities not exceeding 5000 mPa·s, spindle 4 for viscosities not exceeding 10,000 mPa·s, spindle 5 for viscosities not exceeding 20,000 mPa·s, spindle 6 for viscosities not exceeding 50,000 mPa·s and spindle 7 for viscosities not exceeding 200,000 mPa·s.

TABLE 2
Test results

| | Example 1 | Example 2 | | Example 4 | Example 5 | |
|---|---|---|---|---|---|---|
| | (comparative | (comparative | | (comparative | (comparative | |
| Composition [wt. %] | example) | example) | Example 3 | example) | example) | Example 6 |
| Sodium lauryl ether sulfate (SLES) | 9 | 8 | 8 | 8 | 9 | 8 |
| Cocamidopropyl betaine (CAPB) | 3 | 3 | 3 | 3.5 | 3 | 3 |
| Cocomonoethanolamide (CMEA) | 0 | 0 | 0 | 0 | 0.5 | 0 |
| C 12/14 glucamide | 0 | 0 | 0 | 0 | 0 | 0.5 |
| (Preparation Example 1) | | | | | | |
| C 16/18 glucamide | 0 | 1 | 0.5 | 0 | 0 | 0 |
| (Preparation Example 2) | | | | | | |
| Cetyl alcohol | 0 | 0 | 0.5 | 0.5 | 0 | 0.5 |
| Salt level | 1.5 | 1.1 | 1.1 | 1.0 | 1.0 | 1.0 |
| Viscosity at 20° C. | 5800 | 6040 | 3000 | 2750 | 6620 | 3390 |
| Viscosity at 40° C. | 930 | 1430 | 2870 | 2930 (clear) | 1160 | 3310 (clear) |
| Appearance at 20° C. | clear | clear | clear | pearlescent | clear | pearlescent |
| Foam behavior | ◯ | (+) | (++) | (+) | (+) | (+) |
| | standard | creamier than | creamier than | better than | creamier | better than the |
| | | standard, fine | standard, very fine | standard but | | standard but |
| | | foam bubbles | stable foam | less creamy | | less creamy |
| | | | | than Example 3, | | than Example 3, |
| | | | | coarser foam | | foam bubbles |
| | | | | bubbles | | finer than |
| | | | | | | Example 4 |
| Sensory properties of the hair, wet | ◯ | (+) | (+) | ◯ | ◯ | ◯ |
| | standard | good wet | good wet | more squeaky | light, better | more squeaky |
| | | combability | combability | than Example 3 | than standard | than Example 3 |
| | | | | on rinsing out | | on rinsing |
| | | | | | | out |
| Sensory properties of the hair, dry | (−) | ◯ | (+) | ◯ | (+) | ◯ |
| | hair dried out | hair highly | soft and | conditioned | hair lightly | conditioned |
| | after drying | conditioned | conditioned | | conditioned | |

As is apparent from the sensory evaluation of the formulation according to the invention (Example 3), the compositions according to the invention exhibit on the one hand improved foaming behavior and on the other hand also an improved conditioning effect in both the wet and the dry state, not only in comparison with a standard shampoo comprising SLES/betaine (Example 1) but also in comparison with a shampoo comprising SLES/betaine/fatty alcohol (Example 4).

It can additionally be seen that N-alkyl-N-acylglucamines according to Preparation Example 2 increase the viscosity at 40° C. and improve the stability at high temperatures. N-Alkyl-N-acylglucamines according to Preparation Example 2 additionally lead to a pronounced conditioning effect (Krafft point of >50° C.) and increase the creaminess of the foam even at a content of less than 1% by weight.

A mixture of an N-alkyl-N-acylglucamine according to Preparation Example 2 and cetyl alcohol (Example 3) is a possibility for an inexpensive, pearlescent, conditioning component in shampoos which leads to an outstanding foam and high viscosity at 40° C. Such a mixture can also be used in a 2 in 1 shampoo which is free of polyquaternium compounds and is silicone-free.

1. A composition comprising:
at least one N-alkyl-N-acylglucamine as component A,
at least one fatty alcohol as component B,
at least one anionic surfactant as component C,
optionally at least one betaine surfactant as component D,
optionally at least one further surfactant as component E,
water as component F, and
optionally at least one further additive as component G.

2. The composition as claimed in claim 1, wherein the at least one N-alkyl-N-acylglucamine is an N-alkyl-N-acylglucamine of formula (I) 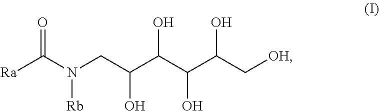
wherein in formula (I)
RₐCO is a linear or branched, saturated or unsaturated C₆-C₂₂-acyl radical and
R_{b} is a C₁-C₄-alkyl radical.

3. The composition as claimed in claim 2, wherein R_{b} is a methyl radical.

4. The composition as claimed in claim 1, wherein RₐCO is a C₁₆-C₁₈-acyl radical.

5. The composition as claimed in claim 1, wherein the radical RₐCO is derived from palmitic acid, stearic acid, oleic acid or linolenic acid.

6. The composition as claimed in claim 2, wherein the at least one N-alkyl-N-acylglucamine is a mixture of
at least one N-alkyl-N-acylglucamine of formula (I) wherein RₐCO denotes a C₁₆-acyl radical, and
at least one N-alkyl-N-acylglucamine of formula (I) wherein RₐCO denotes a C₁₈-acyl radical.

7. The composition as claimed in claim 1, wherein RₐCO is a C₁₂-C₁₄-acyl radical.

8. The composition as claimed in claim 1, wherein the composition comprises an alkyl sulfate and/or an alkyl ether sulfate as the anionic surfactant.

9. The composition as claimed in claim 1, wherein the composition comprises an alkyl sulfate and/or an alkyl ether sulfate as the anionic surfactant and a betaine surfactant.

10. The composition as claimed in claim 1, wherein the composition comprises a linear C₈-C₂₀-alkyl sulfate and/or a linear C₈-C₂₀-alkyl ether sulfate as the anionic surfactant.

11. The composition as claimed in claim 10, wherein the composition comprises lauryl sulfate and/or a lauryl ether sulfate as the anionic surfactant.

12. The composition as claimed in claim 11, wherein the composition comprises sodium lauryl ether sulfate as the anionic surfactant.

13. The composition as claimed in claim 1, wherein the composition comprises an acylamidopropyl betaine or an alkyl betaine as component D.

14. The composition as claimed in claim 13, wherein the composition comprises cocamidopropyl betaine as component D.

15. The composition as claimed in claim 1, wherein the at least one fatty alcohol is a C₁₆-C₂₂-fatty alcohol or a mixture thereof.

16. The composition as claimed in claim 1, wherein the at least one fatty alcohol is selected from the group consisting of cetyl alcohol, stearyl alcohol, behenyl alcohol and a mixture thereof.

17. The composition as claimed in claim 1, wherein the at least one fatty alcohol is selected from the group consisting of cetyl alcohol, stearyl alcohol and a mixture thereof.

18. The composition as claimed in claim 1, wherein the ratio N-alkyl-N-acylglucamine:fatty alcohol is from 20:80 to 80:20.

19. The composition as claimed in claim 1, wherein the at least one component G is selected from the group consisting of preservatives, fragrances, colorants, surfactants, cationic polymers, thickeners and gelling agents, pigments, antimicrobial and biogenic active ingredients, moisturizers, stabilizers, acids and alkalis.

20. The composition as claimed in claim 1, with the proviso that the composition does not comprise cationic polymers and/or cationic surfactants.

21. The composition as claimed in claim 1, with the proviso that the composition does not comprise silicones.

22. The composition as claimed in claim 1, comprising:
from 0.1 to 5.0% by weight, based on the composition, of component A,
from 0.1 to 5.0% by weight, based on the composition, of component B,
from 5.0 to 20.0% by weight, based on the composition, of component C,
from 0 to 10.0% by weight, based on the composition, of component D.

23. A shampoo for greasy hair, dry hair, damaged hair, or hair with dandruff, in the form of a color shampoo, baby shampoo or sport shampoo comprising the composition as claimed in claim 1.

24. A hair care method, wherein the hair is brought into contact with a composition comprising:
at least one N-alkyl-N-acylglucamine as component A,
at least one fatty alcohol as component B,
at least one anionic surfactant as component C,
optionally at least one betaine surfactant as component D,
optionally at least one further surfactant as component E,
water as component F, and
optionally at least one further additive as component G.

25. The method claimed in claim 24 for improving the foaming behavior and/or for improving the conditioning effect.

26. A method for producing a composition comprising:
at least one N-alkyl-N-acylglucamine as component A,
at least one fatty alcohol as component B,
at least one anionic surfactant as component C,
optionally at least one betaine surfactant as component D,
optionally at least one further surfactant as component E,
water as component F, and
optionally at least one further additive as component G wherein components A, B, C, F and optionally D, E, G are brought into contact with one another.
